(19) United States
(12) Patent Application Publication
(10) Pub. No.: US 2022/0061688 A1
(43) Pub. Date: Mar. 3, 2022

(54) MONITORING METHOD AND DEVICE

(71) Applicant: SHENZHEN MINDRAY BIO-MEDICAL ELECTRONICS CO., LTD., Shenzhen (CN)

(72) Inventors: Yingjie JIA, Shenzhen (CN); Haoyu JIANG, Shenzhen (CN); Wenyu YE, Shenzhen (CN); Ping YANG, Shenzhen (CN); Xianliang HE, Shenzhen (CN)

(73) Assignee: SHENZHEN MINDRAY BIO-MEDICAL ELECTRONICS CO., LTD., Shenzhen (CN)

(21) Appl. No.: 17/460,285

(22) Filed: Aug. 29, 2021

(30) Foreign Application Priority Data
Aug. 28, 2020 (CN) 202010885461.7

(51) Int. Cl. A61B 5/0245; A61B 5/00

(52) U.S. Cl. CPC A61B 5/02455; A61B 5/7221; A61B 2505/03; A61B 5/7246; A61B 5/7267

(57) ABSTRACT

Embodiments of the disclosure provide a monitoring method and device. The method includes: obtaining a physiological signal; performing waveform detection on the physiological signal to determine a target waveform position sequence; performing waveform classification on a physiological signal segment corresponding to the target waveform position sequence, to determine a waveform type of each physiological signal segment; performing anomaly detection on classified physiological signal segments by using at least two preset anomaly detection methods, and generating a target alarm event sequence according to detection results of the at least two anomaly detection methods; and outputting the target alarm event sequence. The method of the embodiments of the disclosure can not only make full use of information about an original physiological signal, but can also take advantage of at least two anomaly detection methods, thereby reducing false alarms and missed alarms and improving alarm accuracy.

### CROSS-REFERENCE TO RELATED APPLICATION

The application claims the benefits of priority of Chinese Application No. 202010885461.7, filed Aug. 28, 2020, the content of which is incorporated by reference in its entirety.

### TECHNICAL FIELD

The disclosure relates to the technical field of medical devices, and specifically to a monitoring method and device.

### BACKGROUND

Monitoring devices can provide medical staff with monitoring data indicating patients' vital signs, so that clinicians can grasp changes in conditions of the patients more comprehensively, visually, and in a timely manner, and an important basis can be provided for formulating treatment plans and emergency treatment, to achieve the best treatment effects. Therefore, the monitoring devices are widely used in an intensive care unit (ICU), a coronary care unit (CCU), an anesthesia operating room, and related clinical departments of a hospital.

An existing monitoring device performs feature extraction on physiological signals collected, and determines physiological parameters according to features extracted from the physiological signals. However, the features are abstraction of the original physiological signals from only some angles, without fully utilizing information of the original physiological signals. Same features may be extracted from physiological signals that indicate different diseases, resulting in false alarms or missed alarms and reducing alarm accuracy. Therefore, the alarm accuracy of the existing monitoring device needs to be further improved.

### SUMMARY

Embodiments of the disclosure provide a monitoring method and device, to solve the problem of low alarm accuracy of an existing monitoring device.

According to a first aspect, an embodiment provides a monitoring method, including:

obtaining a physiological signal;

performing waveform detection on the physiological signal to determine a target waveform position sequence;

performing waveform classification on a physiological signal segment corresponding to the target waveform position sequence, to determine a waveform type of each physiological signal segment corresponding to the target waveform position sequence;

performing anomaly detection on classified physiological signal segments by using at least two preset anomaly detection methods, and generating a target alarm event sequence according to detection results of the at least two anomaly detection methods, where an alarm event in the target alarm event sequence is an alarm event determined according to an anomalous physiological signal segment in the classified physiological signal segments; and

outputting the target alarm event sequence.

According to a second aspect, an embodiment provides a monitoring method, including:

obtaining a physiological signal;

performing waveform detection on the physiological signal by using a preset waveform detection method, to determine a target waveform position sequence;

performing, by using a preset waveform classification method, waveform classification on a physiological signal segment corresponding to the target waveform position sequence, to determine a waveform type of each physiological signal segment corresponding to the target waveform position sequence;

performing anomaly detection on classified physiological signal segments by using a preset anomaly detection method, and generating a target alarm event sequence according to a detection result of the anomaly detection method, where an alarm event in the target alarm event sequence is an alarm event determined according to an anomalous physiological signal segment in the classified physiological signal segments; and

outputting the target alarm event sequence,

where at least one of a number of preset waveform detection methods, a number of preset waveform classification methods, and a number of preset anomaly detection methods is at least two.

According to a third aspect, an embodiment provides a monitoring device, including:
a signal acquisition circuit configured to obtain a physiological signal;

an output apparatus configured to output an alarm event;
a memory configured to store a program; and

a processor configured to execute the program stored in the memory, to implement the monitoring method according to any of the embodiments of the disclosure.

According to a fourth aspect, an embodiment provides a computer-readable storage medium, including a program, the program being executable by a processor to implement the monitoring method according to any of the embodiments of the disclosure.

According to the monitoring method and device of the foregoing embodiments, waveform detection and classification are performed on the physiological signal, anomaly detection is performed on the classified physiological signal segments by using the at least two preset anomaly detection methods, and the target alarm event sequence is generated according to the detection results of the at least two anomaly detection methods. This not only makes full use of information about an original physiological signal, but can also integrate advantages of the at least two anomaly detection methods, thereby reducing false alarms and missed alarms and improving alarm accuracy.

### BRIEF DESCRIPTION OF THE DRAWINGS

FIG. 1 is a schematic structural diagram of a monitoring device according to an embodiment;

FIG. 2 is a schematic structural diagram of a monitoring device according to another embodiment;

FIG. 3 is a flowchart of a monitoring method according to an embodiment;

FIG. 4 is a schematic structural diagram of an artificial intelligence waveform detection model according to an embodiment;

FIG. 5 is a flowchart of a waveform detection method according to an embodiment;

FIG. 6 is a flowchart of a waveform detection method according to another embodiment;

FIG. 7 is a schematic structural diagram of an artificial intelligence waveform classification model according to an embodiment;

FIG. 8 is a flowchart of a waveform classification method according to an embodiment;

FIG. 9 is a flowchart of a waveform classification method according to another embodiment;

FIG. 10 is a schematic structural diagram of an artificial intelligence alarm model according to an embodiment;

FIG. 11 is a flowchart of an anomaly detection method according to an embodiment;

FIG. 12 is a flowchart of an anomaly detection method according another embodiment;

FIG. 13 is a schematic architectural diagram of a priority model according to an embodiment; and

FIG. 14 is a schematic structural diagram of a monitoring device according to still another embodiment.

### DETAILED DESCRIPTIONS

The disclosure will be further described in detail below through specific implementations in conjunction with the accompanying drawings. Associated similar element reference numerals are used for similar elements in different implementations. In the following implementations, many details are described such that the disclosure may be better understood. However, it may be effortlessly appreciated by a person skilled in the art that some of the features may be omitted, or may be substituted by other elements, materials, and methods in different cases. In certain cases, some operations involved in the disclosure are not displayed or described in the specification, which is to prevent a core part of the disclosure from being obscured by too much description. Moreover, for a person skilled in the art, the detailed description of the involved operations is not necessary, and the involved operations can be thoroughly understood according to the description in the specification and general technical knowledge in the art.

In addition, the characteristics, operations, or features described in the specification may be combined in any appropriate manner to form various implementations. Meanwhile, the steps or actions in the method description may also be exchanged or adjusted in order in a way that is obvious to a person skilled in the art. Therefore, the various orders in the specification and the accompanying drawings are merely for the purpose of clear description of a certain embodiment and are not meant to be a necessary order unless it is otherwise stated that a certain order must be followed.

The serial numbers themselves for the components herein, for example, “first” and “second”, are merely used to distinguish described objects, and do not have any sequential or technical meaning. Moreover, as used in the disclosure, “connection” or “coupling”, unless otherwise stated, includes both direct and indirect connections (couplings).

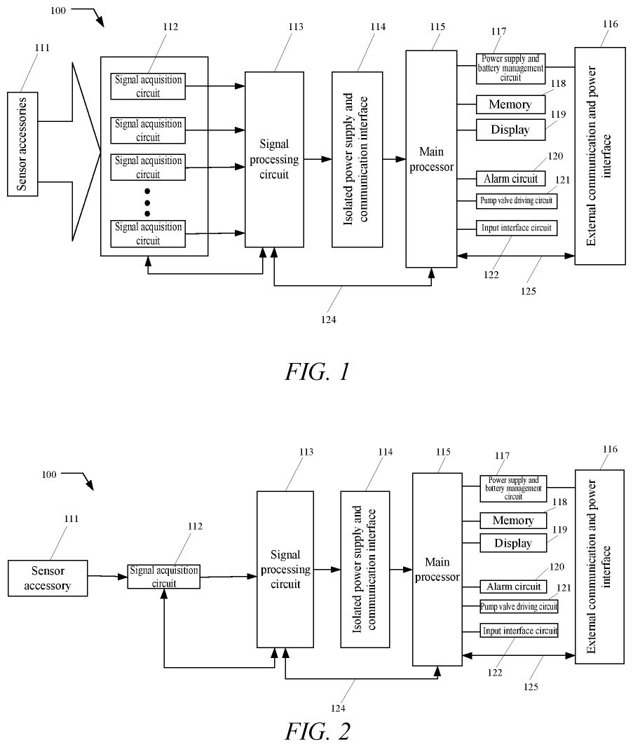

As shown in FIG. 1, a schematic structural diagram of a monitoring device 100 that can be used for multi-parameter monitoring is provided. The monitoring device 100 may have an independent housing, and a sensor interface area may be arranged on a housing panel. A plurality of sensor interfaces may be integrated in the sensor interface area and configured to be connected to various external physiological parameter sensor accessories 111. The housing panel may further include a small IXD display area, a display 119, an input interface circuit 122, an alarm circuit 120 (such as an LED alarm area), and the like. The monitoring device 100 may have an external communication and power interface 116 for communicating with a host and draw power from the host. The monitoring device 100 may also support a build-out parameter module. The parameter module may be plugged in to form a plug-in monitoring device 100 host, and is used as a part of the monitoring device 100. Alternatively, the host may be connected by means of a cable, and the build-out parameter module is used as an external accessory of the monitoring device 100.

An internal circuit of the monitoring device 100 is placed in the housing. As shown in FIG. 1, the internal circuit includes signal acquisition circuits 112 corresponding to at least two physiological parameters, a front-end signal processing circuit 113, and a main processor 115. The signal acquisition circuits 112 may be selected from an electrocardiogram circuit, a respiration circuit, a body temperature circuit, a blood oxygen circuit, a non-invasive blood pressure circuit, an invasive blood pressure circuit, and the like. These signal acquisition circuits 112 are electrically connected to respective sensor interfaces, so as to be electrically connected to the sensor accessories 111 corresponding to different physiological parameters. An output ends of the signal acquisition circuits are coupled to the front-end signal processing circuit 113, a communication port of the front-end signal processing circuit 113 is coupled to the main processor 115, and the main processor 115 is electrically connected to the external communication and power interface 116. The sensor accessories 111 and the signal acquisition circuits 112 corresponding to various physiological parameters may use general-purpose circuits in the prior art. The front-end signal processing circuit 113 completes sampling and analog-to-digital conversion of output signals of the signal acquisition circuits 112, and outputs a control signal to control a measurement process of a physiological signal. These parameters include but are not limited to: parameters such as electrocardiogram, respiration, body temperature, blood oxygen, non-invasive blood pressure, and invasive blood pressure. The front-end signal processing circuit 113 may be implemented by using a single-chip microcomputer or other semiconductor devices, for example, a mixed-signal single-chip microcomputer such as LPC2136 by PHLIPS or ADuC7021 by ADI, or may be implemented by using an ASIC or a FPGA. The front-end signal processing circuit 113 may be powered by an isolated power supply, and data sampled may be sent to the main processor 115 through an isolated communication interface after simple processing and packetization. For example, the front-end signal processing circuit 113 may be coupled to the main processor 115 through an isolated power supply and communication interface 114. A reason for which the front-end signal processing circuit 113 is powered by an isolated power supply is that a DC/DC power supply isolated by a transformer has a function of isolating a patient from a power supply device, with main purposes including: 1. isolating the patient, and enabling an application part to be floating by means of the isolation transformer, so that a leakage current of the patient is small enough; and 2. preventing voltage or energy during defibrillation or electrotome application from affecting a board card and a device of an intermediate circuit such as a main control board (guaranteed by a creepage distance and an electrical clearance). Certainly, the front-end signal processing circuit 113 may alternatively be connected to the main processor 115 by means of a cable 124. The main processor 115 completes calculation of the physiological parameter, and sends a calculation result and waveform of the parameter to the host (such as a host with a display, a PC, and a central station) through the external communication and power interface 116. The main processor 115 may be connected to the external communication and power interface 116 by means of a cable 125, to perform communication and/or draw power. The monitoring device 100 may further include a power supply and battery management circuit 117. The power supply and battery management circuit 117 draws power from the host through the external communication and power interface 116, and supplies power to the main processor 115 after processing such as rectification and filtering. The power supply and battery management circuit 117 may further monitor, manage, and protect the power drawn from the host through the external communication and power interface 116. The external communication and power interface 116 may be one of or a combination of the Ethernet, a token ring, a token bus, and a local area network interface composed of a fiber distributed data interface (FDDI) for a backbone network of these three networks, or may be one of or a combination of wireless interfaces such as infrared, Bluetooth, Wi-Fi, and WMTS communication interfaces, or may be one of or a combination of wired data connection interfaces such as RS232 and USB interfaces. The external communication and power interface 116 may also be one of a wireless data transmission interface and a wired data transmission interface or a combination thereof. The host may be any computer device such as a host of the monitoring device 100, an electrocardiograph machine, an ultrasonic diagnosis instrument, or a computer, and can form the monitoring device 100 once installed with matching software. The host may alternatively be a communication device such as a mobile phone, and the monitoring device 100 sends data to a Bluetooth-enabled mobile phone by using a Bluetooth interface, so as to implement remote transmission of data. The main processor 115 may be further configured to detect the physiological signals acquired by the signal acquisition circuits 112, and output alarm information when an anomaly is detected. The alarm circuit 120 and the display 119 may be used as an output module to output the alarm information. For example, the generated alarm information may be displayed on the display 119, or the alarm circuit 120 may emit an alarm sound for prompting. A memory 118 may store intermediate and final data of the monitoring device 100, and store program instructions or code executed by the main processor 115 and the like. If the monitoring device 100 has a function of blood pressure measurement, the monitoring device may further include a pump valve driving circuit 121. The pump valve driving circuit 121 is configured to perform inflation or deflation operations under the control of the main processor 115.

The monitoring device 100 shown in FIG. 1 is a monitoring device for multi-parameter monitoring. Alternatively, the monitoring device 100 may be a monitoring device for a single physiological parameter. FIG. 2 is an example of a monitoring device for a single physiological parameter. For the same content, reference may be made to the content of FIG. 1 described above, and details are not described herein again.

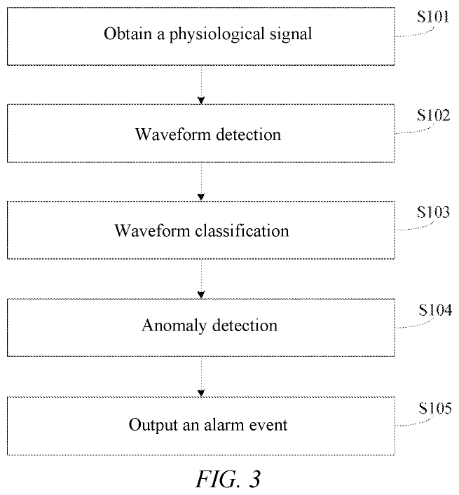

As shown in FIG. 3, an embodiment of the disclosure provides a monitoring method, which can be applied to the monitoring device shown in FIG. 1 or FIG. 2, so as to improve alarm accuracy of the monitoring device. As shown in FIG. 3, the monitoring method provided in this embodiment may include the following steps:

S101: Obtain a physiological signal.

The physiological signal in this embodiment may be an original signal acquired by a signal acquisition circuit using a sensor accessory, or may be a signal generated after general preprocessing of the acquired original signal. General preprocessing may include, for example, signal filtering processing, lead-off processing, signal denoising processing, signal saturation processing, and signal normalization processing. The signal normalization processing may refer to normalizing a sampling rate and resolution of a physiological signal to a preset value. In addition, for a multi-channel physiological signal, various channels of physiological signals may be arranged in a clinically common arrangement order. Taking an electrocardiogram signal as an example, resolution may be indiscriminately adjusted to 200 Lsb/mV, a sampling rate may be indiscriminately adjusted to 250 Hz, and various leads may be arranged in the order of I\II\III\aVR\aVL\aVF\V1 to V6. The physiological signal in this embodiment is a continuous physiological signal, rather than a discrete parameter value, thereby avoiding reduction in alarm accuracy due to information loss that occurs in an extraction process from the continuous physiological signal to the discrete parameter value.

The physiological signal in this embodiment includes, but is not limited to, an electrocardiogram signal, a respiratory signal, a body temperature signal, a blood oxygen signal, a blood pressure signal, etc. The electrocardiogram signal includes but is not limited to a signal acquired by using a lead system such as a 3-lead, 5-lead, or 12-lead system, and the blood pressure signal includes but is not limited to a signal acquired by using a cuff-type blood pressure acquisition system.

S102: Waveform detection. Waveform detection is performed on the physiological signal to determine a target waveform position sequence.

After the physiological signal is obtained, typical waveforms included in the physiological signal are detected, and positions of the typical waveforms in the physiological signal are determined, so as to generate the target waveform position sequence. For an electrocardiogram signal, QRS waveform detection may be performed. For example, the Tompkins algorithm may be used to perform waveform detection on the electrocardiogram (ECG) signal.

In an optional implementation, the physiological signal may be divided into physiological signal segments of a preset length, and then it is determined whether each physiological signal segment includes a typical waveform. If the physiological signal segment includes a typical waveform, a label of the physiological signal segment is set to 1; otherwise, the label of the physiological signal segment is set to 0, so that the target waveform position sequence is generated. It can be understood that the physiological signal may be divided in a partially overlapping manner.

S103: Waveform classification. Waveform classification is performed on a physiological signal segment corresponding to the target waveform position sequence, to determine a waveform type of each physiological signal segment corresponding to the target waveform position sequence.

The typical waveform detected from the physiological signal is classified, to determine the waveform type of each typical waveform. For example, the electrocardiogram signal may be classified into types such as sinus heartbeat, supraventricular heartbeat, nodal heartbeat, and ventricular heartbeat.

S104: Anomaly detection. Anomaly detection is performed on classified physiological signal segments by using at least two preset anomaly detection methods, and a target alarm event sequence is generated according to detection results of the at least two anomaly detection methods, where an alarm event in the target alarm event sequence is an alarm event determined according to an anomalous physiological signal segment in the classified physiological signal segments.

Anomaly detection is performed on the classified physiological signal segments, and an alarm event is generated according to a detected anomalous physiological signal segment. A single anomaly detection method inevitably has certain limitations. However, in this embodiment, the at least two anomaly detection methods are used to perform anomaly detection, so that advantages of the two methods can be fully utilized and integrated to improve alarm accuracy.

S105: Output the target alarm event sequence.

In this embodiment, the target alarm event sequence may be output by using an output module, such as a display, a speaker, or a signal light, of the monitoring device. For example, the target alarm event sequence may be displayed on the display; or the target alarm event sequence may be broadcast by using the speaker; or different types of alarm events in the target alarm event sequence may be prompted by using different signal lights.

When the target alarm event sequence includes a plurality of alarm events, the alarm events may be sorted according to generation time of the alarm events, and then output based on chronological order; or confidence levels, urgency, and importance of the alarm events may be comprehensively evaluated, to determine priorities of the alarm events, and the alarm events are output in descending order of priorities.

Optionally, a select button may alternatively be provided for a user to choose whether to output the target alarm event sequence in chronological order or in priority order.

According to the monitoring method provided in this embodiment, waveform detection and classification are performed on the physiological signal, anomaly detection is performed on the classified physiological signal segments by using the at least two preset anomaly detection methods, and the target alarm event sequence is generated according to the detection results of the at least two anomaly detection methods, thereby implementing a monitoring alarm. The processes of waveform detection, waveform classification, and anomaly detection are all performed on the physiological signal, so that information about the original physiological signal is fully utilized. In addition, the at least two anomaly detection methods are used to perform anomaly detection, so that advantages of the two methods can be fully utilized and integrated. Therefore, the monitoring method provided in this embodiment can reduce false alarms and missed alarms, thereby improving alarm accuracy.

On the basis of the foregoing embodiment, waveform detection, waveform classification, and anomaly detection are separately described in detail below.

First, several specific embodiments are used to describe in detail how to perform waveform detection. In order to avoid reduction in accuracy of waveform detection due to limitations of a single waveform detection method, in this embodiment, waveform detection is performed on a physiological signal by using at least two preset waveform detection methods, and a target waveform position sequence is determined according to detection results of the at least two waveform detection methods. For example, two, three, or more than three waveform detection methods may be used to perform waveform detection, and a specific number of the waveform detection methods may be set according to actual needs, for example, may be determined according to a detection accuracy requirement and/or a processing capability of the monitoring device. For example, in the following description, two different waveform detection methods, namely, a first waveform detection method and a second waveform detection method, are used to perform waveform detection. For implementation of a case of using three or more waveform detection methods to perform waveform detection, reference may be made to the case of using the two methods.

The first waveform detection method and the second waveform detection method in the at least two waveform detection methods are different methods. In an optional implementation, when one of the first waveform detection method and the second waveform detection method is to perform waveform detection on the physiological signal based on a preset detection threshold according to at least one of an amplitude, a slope, and an interval of the physiological signal, the other method may be to perform waveform detection on the physiological signal by using a pre-trained artificial intelligence waveform detection model, where the artificial intelligence waveform detection model is trained based on a physiological signal annotated with a waveform position sequence. A training set of the artificial intelligence waveform detection model is composed of the physiological signal annotated with the waveform position sequence, and the training set may be constructed in the following manner: A physiological signal of at least two physiological cycles is captured, an appropriate threshold is set (for an electrocardiogram signal, a width of a typical QRS wave, that is, 120 ms may be set) to segment the physiological signal. If a physiological signal segment includes most of a QRS wave, a label of the physiological signal segment is set to 1; otherwise, the label is set to 0.

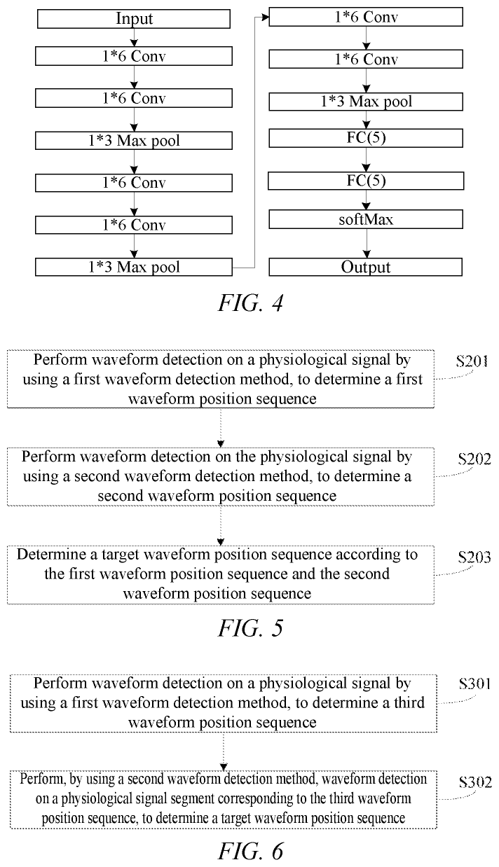

Referring to FIG. 4, an artificial intelligence waveform detection model using a deep convolutional neural network is provided. As shown in FIG. 4, the model includes a plurality of convolutional layers (Cony), a maximum pooling layer (Max pool), and a fully connected layer (FC). An input is a physiological signal, and an output is a 0-1 sequence representing the presence or absence of a typical waveform such as a QRS wave.

In a case of using three or more waveform detection methods, the plurality of waveform detection methods may be, for example, using a plurality of different pre-trained artificial intelligence waveform detection models to perform waveform detection on a physiological signal separately.

Referring to FIG. 5, in an optional implementation, performing waveform detection on the physiological signal by using at least two preset waveform detection methods, and determining the target waveform position sequence according to detection results of the at least two waveform detection methods may include the following steps.

S201: Perform waveform detection on the physiological signal by using the first waveform detection method, to determine a first waveform position sequence.

S202: Perform waveform detection on the physiological signal by using the second waveform detection method, to determine a second waveform position sequence.

It should be noted that this embodiment does not limit an execution order of step S201 and step S202, and they may be performed simultaneously or successively.

S203: Determine the target waveform position sequence according to the first waveform position sequence and the second waveform position sequence.

In this embodiment, after the first waveform detection method and the second waveform detection method are separately used to perform waveform detection on the physiological signal, to generate the first waveform position sequence and the second waveform position sequence, the two waveform position sequences may be integrated according to a confidence level, a matching degree, or a user instruction.

In an optional implementation, integrating the two waveform position sequences according to a confidence level may include:

if a confidence level of the first waveform detection method is greater than a confidence level of the second waveform detection method, determining that the target waveform position sequence is the first waveform position sequence; or

if a confidence level of the first waveform detection method is less than or equal than a confidence level of the second waveform detection method, determining that the target waveform position sequence is the second waveform position sequence.

A confidence level of a waveform detection method may be determined according to detection accuracy of the waveform detection method in an offline database, and preset in the monitoring device. In addition, in a monitoring process, the confidence levels of the two waveform detection methods may be updated depending on a user's confirmation of a waveform detection result.

Specifically, updating the confidence levels of the waveform detection methods may include:

updating the confidence level of the first waveform detection method according to a proportion of a number of confirmed waveform positions in the first waveform position sequence; and

updating the confidence level of the second waveform detection method according to a proportion of a number of confirmed waveform positions in the second waveform position sequence.

For example, a percentage of the number of waveform positions confirmed by the user in a total number of waveforms included in the first waveform position sequence may be used as the confidence level of the first waveform detection method; and a percentage of the number of waveform positions confirmed by the user in a total number of waveforms included in the second waveform position sequence may be used as the confidence level of the second waveform detection method.

In an optional implementation, integrating the two waveform position sequences according to a matching degree may include:

incorporating matching waveform positions in the first waveform position sequence and the second waveform position sequence into the target waveform position sequence; and/or

for any physiological signal segment in the physiological signal, when a first waveform position that is in the first waveform position sequence and corresponds to the physiological signal segment does not match a second waveform position that is in the second waveform position sequence and corresponds to the physiological signal segment, matching the physiological signal segment, the first waveform position, and the second waveform position with a historical waveform database, where the historical waveform database stores a correspondence between a physiological signal segment and a corresponding detected waveform position;

incorporating a successful match in the first waveform position and the second waveform position into the target waveform position sequence; and determining a failed match in the first waveform position and the second waveform position as a false detection.

Matching can be understood as being the same or as a difference meeting a preset condition. For example, waveform positions presented in both the first waveform position sequence and the second waveform position sequence are incorporated into the target waveform position sequence. For a waveform position presented only in the first waveform position sequence or only in the second waveform position sequence, a physiological signal segment corresponding to the waveform position may be captured for matching in the historical waveform database.

Optionally, physiological signal segments corresponding to the waveform positions incorporated into the target waveform position sequence may further be added to the historical waveform database.

In an optional implementation, integrating the two waveform position sequences according to a user instruction may include: determining, according to the user instruction, to output the first waveform position sequence or output the second waveform position sequence. For example, a select button may be provided for the user to choose whether to output the first waveform position sequence or output the second waveform position sequence.

According to the monitoring method provided in this embodiment, on the basis of the foregoing embodiment, the first waveform detection method and the second waveform detection method are separately used to perform waveform detection on the physiological signal, and the waveform position sequences generated by using the two waveform detection methods are integrated, so that accuracy of waveform detection is improved, and alarm accuracy can be further effectively improved.

With reference to the foregoing method, in the case of using three or more waveform detection methods, after a plurality of waveform position sequences are separately determined, the plurality of waveform position sequences may be integrated according to a confidence level, a matching degree, or a user instruction. For example, the target waveform position sequence may be determined as a waveform position sequence detected by using a waveform detection method with a highest confidence level; or one of the plurality of waveform position sequences may be output according to the user instruction; or matching waveform positions in the plurality of waveform position sequences may be directly incorporated into the target waveform position sequence, while for mismatching waveform positions, matching is performed in the historical waveform database.

Referring to FIG. 6, in another optional implementation, performing waveform detection on the physiological signal by using at least two preset waveform detection methods, and determining the target waveform position sequence according to detection results of the at least two waveform detection methods may include the following steps.

S301: Perform waveform detection on the physiological signal by using the first waveform detection method, to determine a third waveform position sequence.

S302: Perform, by using the second waveform detection method, waveform detection on a physiological signal segment corresponding to the third waveform position sequence, to determine the target waveform position sequence.

Sensitivity of the first waveform detection method is higher than sensitivity of the second waveform detection method, and specificity of the second waveform detection method is higher than specificity of the first waveform detection method.

In this embodiment, sensitivity refers to a probability that physiological signal segments including typical waveforms are not missed during waveform detection, and a higher sensitivity indicates a lower probability of missed detection. Specificity refers to a probability of no false detection during waveform detection, and a higher specificity indicates a lower probability of false detection. For the waveform detection method based on the preset detection threshold, sensitivity and specificity may be adjusted by changing the preset detection threshold. For the waveform detection method based on the artificial intelligence waveform detection model, sensitivity and specificity may be adjusted by adjusting a number and proportion of samples in the training set for training the artificial intelligence waveform detection model.

In an optional implementation, a waveform position sequence obtained by using the second waveform detection method to perform waveform detection on the physiological signal segment corresponding to the third waveform position sequence may be determined as the target waveform position sequence.

In another optional implementation, performing, by using the second waveform detection method, waveform detection on a physiological signal segment corresponding to the third waveform position sequence, to determine the target waveform position sequence may include:

for any physiological signal segment in the third waveform position sequence, detecting the physiological signal segment by using the second waveform detection method, to obtain a second waveform position, and determining a target waveform position of the physiological signal segment according to the second waveform position and a first waveform position that is obtained by detecting the physiological signal segment by using the first waveform detection method; and determining the target waveform position sequence according to target waveform positions of physiological signal segments in the third waveform position sequence.

According to the monitoring method provided in this embodiment, on the basis of the foregoing embodiment, the first waveform detection method with a higher sensitivity is first used for waveform detection, to find all physiological signal segments that may include typical waveforms, so that missed detection can be effectively avoided; and then the second waveform detection method with a higher specificity is used to double-check the physiological signal segments found by using the first waveform detection method, so that false detection can be effectively avoided. The advantages of the two waveform detection methods complement each other, which improves accuracy of waveform detection, and can further improve alarm accuracy.

With reference to the foregoing method, in the case of using three or more waveform detection methods, the plurality of waveform detection methods may be first sorted in descending order of sensitivity and in ascending order of specificity, and then are successively used for waveform detection.

Then, several specific embodiments are used to describe in detail how to perform waveform classification. In order to avoid reduction in accuracy of waveform classification due to limitations of a single waveform classification method, in this embodiment, at least two preset waveform classification methods are used to perform waveform classification on a physiological signal segment corresponding to a target waveform position sequence, and a waveform type of each physiological signal segment corresponding to the target waveform position sequence is determined according to classification results of the at least two preset waveform classification methods. For example, two, three, or more than three waveform classification methods may be used to perform waveform classification, and a specific number of the waveform classification methods may be set according to actual needs, for example, may be determined according to a classification accuracy requirement and/or a processing capability of the monitoring device. For example, in the following description, two different waveform classification methods, namely, a first waveform classification method and a second waveform classification method, are used to perform waveform detection. For implementation of a case of using three or more waveform classification methods to perform waveform classification, reference may be made to the case of using the two methods.

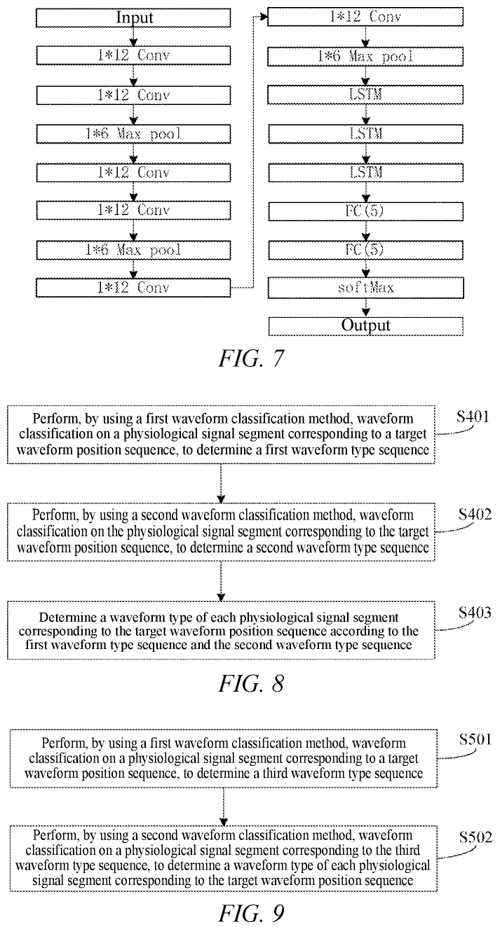

The first waveform classification method and the second waveform classification method in the at least two waveform classification methods are different methods. In an optional implementation, when one of the first waveform classification method and the second waveform classification method is to perform waveform classification on the physiological signal segment based on a preset classification threshold according to at least one of an amplitude, a slope, and an interval, the other method is to perform waveform classification on the physiological signal segment by using a pre-trained artificial intelligence waveform classification model, where the artificial intelligence waveform classification model is trained based on a physiological signal segment annotated with a waveform type. Referring to FIG. 7, an artificial intelligence waveform classification model using a deep convolutional neural network is provided. As shown in FIG. 7, the model includes a plurality of convolutional layers (Cony), a maximum pooling layer (Max pool), and a fully connected layer (FC). In addition, considering that waveform classification generated during a long period of monitoring depends on timing characteristics, a long short-term memory (LSTM) neural network is introduced. An input is a physiological signal segment, and an output is a waveform type.

In a case of using three or more waveform classification methods, the plurality of waveform classification methods may be, for example, using a plurality of different pre-trained artificial intelligence waveform classification models to perform waveform classification on a physiological signal segment separately.

Referring to FIG. 8, in an optional implementation, performing, by using at least two preset waveform classification methods, waveform classification on the physiological signal segment corresponding to the target waveform position sequence, and determining the waveform type of each physiological signal segment corresponding to the target waveform position sequence according to classification results of the at least two preset waveform classification methods may include the following steps.

S401: Perform, by using the first waveform classification method, waveform classification on the physiological signal segment corresponding to the target waveform position sequence, to determine a first waveform type sequence.

S402: Perform, by using the second waveform classification method, waveform classification on the physiological signal segment corresponding to the target waveform position sequence, to determine a second waveform type sequence.

It should be noted that this embodiment does not limit an execution order of step S401 and step S402, and they may be performed simultaneously or successively.

S403: Determine the waveform type of each physiological signal segment corresponding to the target waveform position sequence according to the first waveform type sequence and the second waveform type sequence.

In this embodiment, after the first waveform classification method and the second waveform classification method are separately used to perform waveform classification on the physiological signal segment, to generate the first waveform type sequence and the second waveform type sequence, the two waveform position sequences may be integrated according to a confidence level, a matching degree, or a user instruction.

In an optional implementation, determining the waveform type of each physiological signal segment corresponding to the target waveform position sequence according to the first waveform type sequence and the second waveform type sequence may include:

if a confidence level of the first waveform classification method is higher than a confidence level of the second waveform classification method, determining that the waveform type of each physiological signal segment corresponding to the target waveform position sequence uses the first waveform type sequence; or

if a confidence level of the first waveform classification method is less than or equal to a confidence level of the second waveform classification method, determining that the waveform type of each physiological signal segment corresponding to the target waveform position sequence uses the second waveform type sequence.

A confidence level of a waveform classification method may be determined according to classification accuracy of the waveform classification method in an offline database, and preset in the monitoring device. In addition, in a monitoring process, the confidence levels of the two waveform classification methods may be updated depending on a user's confirmation of a waveform classification result.

Specifically, updating the confidence levels of the waveform classification methods may include:

updating the confidence level of the first waveform classification method according to a proportion of a number of confirmed waveform types in the first waveform type sequence; and

updating the confidence level of the second waveform classification method according to a proportion of a number of confirmed waveform types in the second waveform type sequence.

In another optional implementation, determining the waveform type of each physiological signal segment corresponding to the target waveform position sequence according to the first waveform type sequence and the second waveform type sequence may include:

determining same waveform types in the first waveform type sequence and the second waveform type sequence as waveform types of corresponding physiological signal segments; and/or

for any physiological signal segment corresponding to the target waveform position sequence, when a first waveform type that is in the first waveform type sequence and corresponds to the physiological signal segment is different from a second waveform type that is in the second waveform type sequence and corresponds to the physiological signal segment, matching the physiological signal segment, the first waveform type, and the second waveform type with a historical waveform type database, where the historical waveform type database stores a correspondence between a physiological signal segment and a corresponding waveform type;

determining a successful match in the first waveform type and the second waveform type as a waveform type of a corresponding physiological signal segment; and determining a failed match in the first waveform type and the second waveform type as a false classification.

According to the monitoring method provided in this embodiment, on the basis of the foregoing embodiment, the first waveform classification method and the second waveform classification method are separately used to perform waveform classification on the physiological signal segment, and the waveform type sequences generated by using the two waveform classification methods are integrated, so that accuracy of waveform classification is improved, which helps improve alarm accuracy.

With reference to the foregoing method, in the case of using three or more waveform classification methods, after a plurality of waveform type sequences are separately determined, the plurality of waveform type sequences may be integrated according to a confidence level, a matching degree, or a user instruction. For example, waveform types of physiological signal segments corresponding to the target waveform position sequence may be determined as a waveform type sequence generated by using a waveform classification method with a highest confidence level; or one of the plurality of waveform type sequences may be output according to the user instruction; or same waveform types in the plurality of waveform type sequences may be determined as waveform types of corresponding physiological signal segments, while for different waveform types, matching is performed in the historical waveform type database.

Referring to FIG. 9, in another optional implementation, performing, by using at least two preset waveform classification methods, waveform classification on the physiological signal segment corresponding to the target waveform position sequence, and determining the waveform type of each physiological signal segment corresponding to the target waveform position sequence according to classification results of the at least two preset waveform classification methods may include the following steps.

S501: Perform, by using the first waveform classification method, waveform classification on the physiological signal segment corresponding to the target waveform position sequence, to determine a third waveform type sequence.

S502: Perform, by using the second waveform classification method, waveform classification on a physiological signal segment corresponding to the third waveform type sequence, to determine the waveform type of each physiological signal segment corresponding to the target waveform position sequence.

Sensitivity of the first waveform classification method is higher than sensitivity of the second waveform classification method, and specificity of the second waveform classification method is higher than specificity of the first waveform classification method.

In an optional implementation, a waveform type sequence obtained by using the second waveform classification method to perform waveform classification on the physiological signal segment corresponding to the third waveform type sequence may be determined as the waveform type of each physiological signal segment corresponding to the target waveform position sequence.

In another optional implementation, performing, by using the second waveform classification method, waveform classification on a physiological signal segment corresponding to the third waveform type sequence, to determine the waveform type of each physiological signal segment corresponding to the target waveform position sequence may include:

for any physiological signal segment in the third waveform type sequence, classifying the physiological signal segment by using the second waveform classification method, to obtain a second waveform type, and determining a target waveform type of the physiological signal segment according to the second waveform type and a first waveform type that is obtained by classifying the physiological signal segment by using the first waveform classification method; and determining the target waveform type sequence according to target waveform types of physiological signal segments in the third waveform type sequence.

According to the monitoring method provided in this embodiment, on the basis of the foregoing embodiment, the first waveform classification method with a higher sensitivity is first used for waveform classification; and then the second waveform classification method with a higher specificity is used to double-check the waveform type output by using the first waveform classification method. The advantages of the two waveform classification methods complement each other, which improves accuracy of waveform classification, and helps improve alarm accuracy.

With reference to the foregoing method, in the case of using three or more waveform classification methods, the plurality of waveform classification methods may be first sorted in descending order of sensitivity and in ascending order of specificity, and then are successively used for waveform classification.

Finally, several specific embodiments are used to describe in detail how to perform anomaly detection. In order to avoid reduction in alarm accuracy due to limitations of a single anomaly detection method, in this embodiment, anomaly detection is performed on classified physiological signal segments by using at least two preset anomaly detection methods, and a target alarm event sequence is generated according to detection results of the at least two anomaly detection methods. For example, two, three, or more than three anomaly detection methods may be used to perform anomaly detection, and a specific number of the anomaly detection methods may be set according to actual needs, for example, may be determined according to an alarm accuracy requirement and/or a processing capability of the monitoring device. For example, in the following description, two different anomaly detection methods, namely, a first anomaly detection method and a second anomaly detection method, are used to perform anomaly detection. For implementation of a case of using three or more anomaly detection methods to perform anomaly detection, reference may be made to the case of using the two methods.

The first anomaly detection method and the second anomaly detection method in the at least two anomaly detection methods are different methods. In an optional implementation, when one of the first anomaly detection method and the second anomaly detection method is to perform anomaly detection on the physiological signal segment based on a preset alarm threshold according to at least one of a waveform type, waveform start and end points, a heart rate, an amplitude, and an interval of the physiological signal segment, the other method is to perform anomaly detection on the physiological signal segment by using a pre-trained artificial intelligence alarm model, where the artificial intelligence alarm model is trained based on a physiological signal segment annotated with an alarm event.

The preset alarm threshold may be determined according to clinical experience and medical guidelines. For example, according to currently and historically detected waveform positions and types, a pattern may be further analyzed, so as to analyze start and end points of each component of a waveform and calculate parameters such as a heart rate, an amplitude, and an interval, and then whether the current data is anomalous is determined by using the preset alarm threshold according to these characteristics. The artificial intelligence alarm model includes, but is not limited to, a deep convolutional neural network, a decision tree, etc. A physiological signal segment is input, and an alarm event is output. A training set of the model may be composed of the physiological signal segment annotated with the alarm event, and the training set may be constructed in the following manner: capturing a physiological signal of at least 10 seconds, and performing per-second annotation according to waveform characteristics of the physiological signal, where if an anomaly is not determined sufficiently in the current second, the current second is annotated as “normal”, and a corresponding alarm event is annotated for an anomalous data segment.

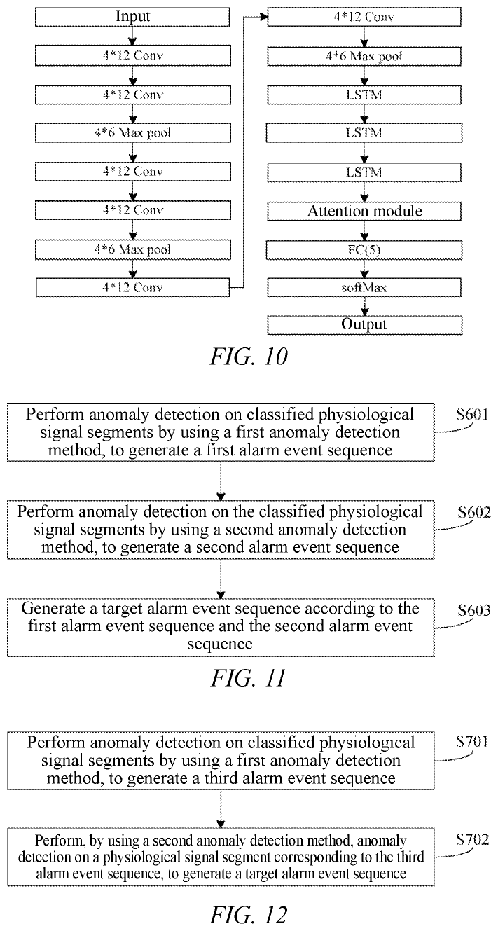

Referring to FIG. 10, an artificial intelligence alarm model using a deep convolutional neural network is provided. As shown in FIG. 10, the model includes a convolutional layer (Cony), a maximum pooling layer (Max pool), and a fully connected layer (FC). In addition, considering that an alarm event generated during a long period of monitoring depends on timing characteristics, a long short-term memory (LSTM) neural network and an attention module are introduced to highlight anomalous information.

In a case of using three or more anomaly detection methods, the plurality of anomaly detection methods may be, for example, using a plurality of different pre-trained artificial intelligence alarm models to perform anomaly detection on a physiological signal segment separately.

Referring to FIG. 11, in an optional implementation, performing anomaly detection on classified physiological signal segments by using at least two preset anomaly detection methods, and generating a target alarm event sequence according to detection results of the at least two anomaly detection methods may include the following steps.

S601: Perform anomaly detection on the classified physiological signal segments by using the first anomaly detection method, to generate a first alarm event sequence.

S602: Perform anomaly detection on the classified physiological signal segments by using the second anomaly detection method, to generate a second alarm event sequence.

It should be noted that this embodiment does not limit an execution order of step S601 and step S602, and they may be performed simultaneously or successively.

S603: Generate a target alarm event sequence according to the first alarm event sequence and the second alarm event sequence.

In this embodiment, after the first anomaly detection method and the second anomaly detection method are separately used to perform anomaly detection on the classified physiological signal segments, to generate the first alarm event sequence and the second alarm event sequence, the two alarm event sequences may be integrated according to a confidence level, a matching degree, or a user instruction.

In an optional implementation, integrating the first alarm event sequence and the second alarm event sequence according to a confidence level may include:

if a confidence level of the first anomaly detection method is greater than a confidence level of the second anomaly detection method, determining that the target alarm event sequence is the first alarm event sequence; or

if a confidence level of the first anomaly detection method is less than or equal to a confidence level of the second anomaly detection method, determining that the target alarm event sequence is the second alarm event sequence.

A confidence level of an anomaly detection method may be determined according to detection accuracy of the anomaly detection method in an offline database, and preset in the monitoring device. In addition, in a monitoring process, the confidence levels of the two anomaly detection methods may be updated depending on a user's confirmation of an anomaly detection result.

Specifically, updating the confidence levels of the anomaly detection methods may include:

updating the confidence level of the first anomaly detection method according to a proportion of a number of confirmed alarm events in the first alarm event sequence; and/or

updating the confidence level of the second anomaly detection method according to a proportion of a number of confirmed alarm events in the second alarm event sequence.

For example, a percentage of the number of alarm events confirmed by the user in a total number of alarm events included in the first alarm event sequence may be used as the confidence level of the first anomaly detection method; and a percentage of the number of alarm events confirmed by the user in a total number of alarm events included in the second alarm event sequence may be used as the confidence level of the second anomaly detection method.

In an optional implementation, integrating the first alarm event sequence and the second alarm event sequence according to a matching degree may include:

incorporating matching alarm events in the first alarm event sequence and the second alarm event sequence into the target alarm event sequence; and/or

for any physiological signal segment in the classified physiological signal segments, when a first alarm event that is in the first alarm event sequence and corresponds to the physiological signal segment does not match a second alarm event that is in the second alarm event sequence and corresponds to the physiological signal segment, matching the physiological signal segment, the first alarm event, and the second alarm event with a historical alarm database, where the historical alarm database stores a correspondence between a physiological signal segment and a corresponding detected alarm event;

incorporating a successful match in the first alarm event and the second alarm event into the target alarm event sequence; and determining a failed match in the first alarm event and the second alarm event as a false alarm.

Matching can be understood as being the same or as a difference meeting a preset condition. For example, alarm events presented in both the first alarm event sequence and the second alarm event sequence are incorporated into the target alarm event sequence for outputting. For an alarm event appearing only in the first alarm event sequence or only in the second alarm event sequence, a physiological signal segment corresponding to the alarm event may be captured for matching in the historical alarm database.

Optionally, the alarm events incorporated into the target alarm event sequence and physiological signal segments corresponding to the alarm events may further be added to the historical alarm database.

In an optional implementation, integrating the two alarm event sequences according to a user instruction may include: determining, according to the user instruction, to output the first alarm event sequence or output the second alarm event sequence. For example, a select button may be provided for the user to choose whether to output the first alarm event sequence or output the second alarm event sequence.

According to the monitoring method provided in this embodiment, on the basis of the foregoing embodiment, the first anomaly detection method and the second anomaly detection method are separately used to perform anomaly detection on the classified physiological signal segments, and the alarm event sequences generated by using the two anomaly detection methods are integrated, so that alarm accuracy be effectively improved.

With reference to the foregoing method, in the case of using three or more anomaly detection methods, after a plurality of alarm event sequences are generated, the plurality of alarm event sequences may be integrated according to a confidence level, a matching degree, or a user instruction. For example, an alarm event sequence generated by using an anomaly detection method with a highest confidence level may be determined as the target alarm event sequence; or matching alarm events in the plurality of alarm event sequences may be incorporated into the target alarm event sequence, while for mismatching alarm events, matching is performed in the historical alarm database; or one of the plurality of alarm event sequences may be output according to the user instruction.

Referring to FIG. 12, in another optional implementation, performing anomaly detection on classified physiological signal segments by using at least two preset anomaly detection methods, and generating a target alarm event sequence according to detection results of the at least two anomaly detection methods may include the following steps.

S701: Perform anomaly detection on the classified physiological signal segments by using the first anomaly detection method, to generate a third alarm event sequence.

S702: Perform, by using the second anomaly detection method, anomaly detection on a physiological signal segment corresponding to the third alarm event sequence, to generate the target alarm event sequence.

Sensitivity of the first anomaly detection method is higher than sensitivity of the second anomaly detection method, and specificity of the second anomaly detection method is higher than specificity of the first anomaly detection method.

In this embodiment, sensitivity refers to a probability that anomalous physiological signal segments are not missed during anomaly detection, and a higher sensitivity indicates a lower probability of missed detection. Specificity refers to a probability of no false detection during anomaly detection, and a higher specificity indicates a lower probability of false detection. For the anomaly detection method based on the preset alarm threshold, sensitivity and specificity may be adjusted by changing the preset alarm threshold. For the anomaly detection method based on the artificial intelligence alarm model, sensitivity and specificity may be adjusted by adjusting a number and proportion of samples in the training set for training the artificial intelligence alarm model.

In an optional implementation, an alarm event sequence generated by using the second anomaly detection method to perform anomaly detection on the physiological signal segment corresponding to the third alarm event sequence may be determined as the target alarm event sequence.

In another optional implementation, performing, by using the second anomaly detection method, anomaly detection on a physiological signal segment corresponding to the third alarm event sequence, to generate the target alarm event sequence may include:

for a physiological signal segment corresponding to any alarm event in the third alarm event sequence, detecting the physiological signal segment by using the second anomaly detection method, to obtain a second alarm event, and determining a target alarm event corresponding to the physiological signal segment according to the second alarm event and a first alarm event that is obtained by detecting the physiological signal segment by using the first anomaly detection method; and determining the target alarm event sequence according to target alarm events that are in the third alarm event sequence and correspond to physiological signal segments.

According to the monitoring method provided in this embodiment, on the basis of the foregoing embodiment, the first anomaly detection method with a higher sensitivity is first used for anomaly detection, to find all possible anomalous physiological signal segments, so that missed alarms can be effectively avoided; and then the second anomaly detection method with a higher specificity is used to double-check the anomalous physiological signal segments found by using the first anomaly detection method, so that false alarms can be effectively avoided. The advantages of the two anomaly detection methods complement each other, which improves alarm accuracy.

With reference to the foregoing method, in the case of using three or more anomaly detection methods, the plurality of anomaly detection methods may be first sorted in descending order of sensitivity and in ascending order of specificity, and then are successively used for anomaly detection.

In order to prevent an important alarm event from being drowned in a large number of alarm events, on the basis of any of the foregoing embodiments, the monitoring method provided in this embodiment further includes measuring alarm value of an alarm event. In this embodiment, the alarm value is measured by priority, a higher priority indicates higher alarm value, and the alarm events are sorted by priority, so as to conveniently present an alarm event with clinical value. Performing anomaly detection on classified physiological signal segments by using preset anomaly detection methods, to generate a target alarm event sequence may specifically include: performing anomaly detection on the classified physiological signal segments by using the preset anomaly detection methods, to generate an alarm event set; for any alarm event in the alarm event set, obtaining a plurality of pieces of priority-related characteristic information of the alarm event; respectively inputting the plurality of pieces of characteristic information to a plurality of corresponding pre-trained alarm priority models, to obtain a plurality of sub-priorities of the alarm event; determining a target priority of the alarm event according to the plurality of sub-priorities of the alarm event; and sorting alarm events in the alarm event set according to target priorities of the alarm events in the alarm event set, to obtain the target alarm event sequence.

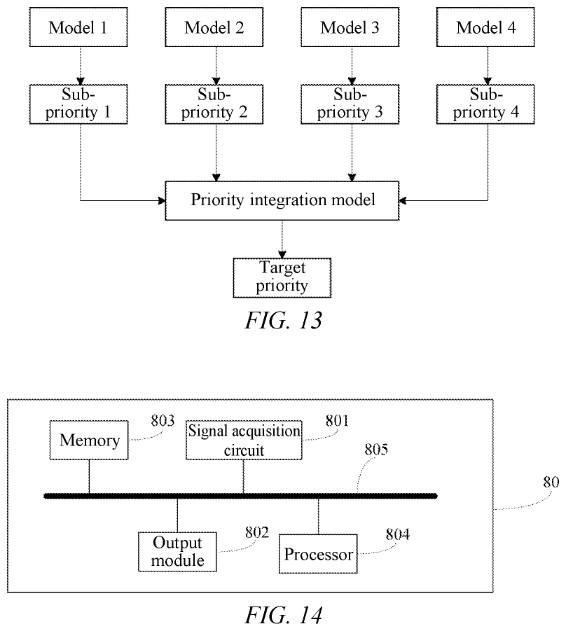

A priority of an alarm event is related to a plurality of factors, such as gender, age, and a disease type of a patient, a physiological parameter value, and an alarm event sequence and its waveform signal within a preset time period before and after a current alarm moment. The priority-related information may be divided into a plurality of different types, such that information of a same type is strongly correlated, and information of different types is weakly correlated. Different alarm priority models are designed for different types of information. Then sub-priorities output by the plurality of alarm priority models are integrated, to determine the target priority of the alarm event. Referring to FIG. 13, FIG. 13 is a schematic architectural diagram of a priority model according to an embodiment, including four alarm priority models. A model 1, a model 2, a model 3, and a model 4 each are used to determine a sub-priority according to a type of priority-related information. The model 1 may be used to determine a sub-priority 1 of an alarm event according to an alarm event sequence within a preset time period before and after a current alarm moment. The model 2 may be used to determine a sub-priority 2 of the alarm event according to a physiological parameter value within the preset time period before and after the current alarm moment. The model 3 may be used to determine a sub-priority 3 of the alarm event according to a waveform signal within the preset time period before and after the current alarm moment. The model 4 may be used to determine a sub-priority 4 of the alarm event according to patient information, such as gender, age, and disease type. A priority integration model is used to determine the target priority of the alarm event according to all the sub-priorities of the alarm event.

On the basis of any of the foregoing embodiments, a signal quality index of the physiological signal may be further determined according to time-frequency domain characteristics of the physiological signal, or determined based on an original physiological signal by using a pre-trained artificial intelligence signal quality evaluation model. Specifically, before waveform detection is performed on the physiological signal, the physiological signal may be analyzed to obtain the signal quality index of the physiological signal.

In an optional implementation, the signal quality index of the physiological signal may be determined according to at least one of an amplitude, a slope, and a power spectrum of the physiological signal. For example, signal quality may be evaluated according to proportions of the parameters such as the amplitude, the slope, and the power spectrum of the physiological signal in a reasonable range. Specifically, the signal quality index of the physiological signal may be determined according to the following formula:

δ=1−(α+β+2*γ)/4

where δ denotes the signal quality index; α denotes a percentage by which the amplitude of the physiological signal exceeds a preset amplitude range, where the preset amplitude range of the signal may be determined according to clinical experience and medical guidelines, the percentage α exceeding this range is calculated, and a may reflect intensity of a low-frequency noise in a saturation section; β denotes a percentage by which the slope of the physiological signal exceeds a preset slope range, where the preset slope range of the physiological signal may be determined according to a reasonable range of a signal difference or a higher-order difference indicated by clinical experience and medical guidelines, the percentage β exceeding this range is calculated, and β may reflect intensity of high-frequency noise interference; and γ denotes a power percentage of the physiological signal beyond the preset frequency range, a spectrum-power distribution graph of the physiological signal may be calculated, the preset frequency range of the physiological signal may be determined according to clinical experience and medical guidelines, the power percentage γ exceeding the preset frequency range is calculated, and γ may comprehensively reflect intensity of high- and low-frequency noise.

In another optional implementation, the signal quality index of the physiological signal may be determined by using a pre-trained artificial intelligence signal quality evaluation model. Specifically, the physiological signal may be input to the pre-trained artificial intelligence signal quality evaluation model, to obtain the signal quality index of the physiological signal, where the artificial intelligence signal quality evaluation model is trained based on a physiological signal annotated with a signal quality index.

A large amount of physiological signal data including different intensities of noise may be collected, and the physiological signal data is annotated with a signal quality index, to establish a physiological signal quality evaluation database. The physiological signal data in the physiological signal quality evaluation database may be normalized, and then a signal quality index label may be annotated. The signal quality index may be a continuous percentage, or may be a discrete sequence. An electrocardiogram signal is taken as an example. For example, quality evaluation may be performed on an electrocardiogram signal segment every 1 second, to annotate a signal quality index. For a data segment of more than 1 second, a signal quality index thereof may be a weighted average of signal quality indexes of all 1-second electrocardiogram signal segments contained therein. In a multi-lead case, a final signal quality index is an average of signal quality indexes on all leads. Then the artificial intelligence signal quality evaluation model is trained based on the established physiological signal quality evaluation database, and the artificial intelligence signal quality evaluation model may be a deep convolutional model. When a trained model is used for signal quality evaluation, only the physiological signal needs to be input to the model, and then the signal quality index can be output.

The signal quality index may be measured by a continuous indicator, or may be measured by a discrete indicator. The signal quality index may be a sequence describing a quality level, such as “signal quality is good”, “signal quality is poor, restricted for use”, and “signal quality is extremely poor, not allowed for use”; or “first-level signal”, “second-level signal”, “third-level signal”, and “four-level signal”. Alternatively, a signal quality index of an extremely poor signal may be set to 0, a signal quality index of a signal that can be used normally may be set to 100, and a signal quality index of a remaining signal may continuously vary between 0 and 100.

After the signal quality index of the physiological signal is determined, the signal quality index of the physiological signal may further be output by using the output module of the monitoring device. For example, the signal quality index may be displayed on a screen to indicate a user to confirm, and prompt the user to improve signal quality.

An embodiment of the disclosure further provides a monitoring method, including:

obtaining a physiological signal;

performing waveform detection on the physiological signal by using a preset waveform detection method, to determine a target waveform position sequence;

performing, by using a preset waveform classification method, waveform classification on a physiological signal segment corresponding to the target waveform position sequence, to determine a waveform type of each physiological signal segment corresponding to the target waveform position sequence;

performing anomaly detection on classified physiological signal segments by using a preset anomaly detection method, and generating a target alarm event sequence according to a detection result of the anomaly detection method, where an alarm event in the target alarm event sequence is an alarm event determined according to an anomalous physiological signal segment in the classified physiological signal segments; and

outputting the target alarm event sequence,

where at least one of a number of preset waveform detection methods, a number of preset waveform classification methods, and a number of preset anomaly detection methods is at least two.

An embodiment of the disclosure further provides a monitoring device, as shown in FIG. 14. As shown in FIG. 14, the monitoring device 80 provided in this embodiment may include: a signal acquisition circuit 801, an output module 802, a memory 803, a processor 804, and a bus 805. The bus 805 is configured to implement connection between various elements.

The signal acquisition circuit 801 obtains a physiological signal by using a sensor accessory connected to a patient.

The output module 802 is configured to output alarm information.

The memory 803 stores a computer program, and when the computer program is executed by the processor 804, the technical solution of any of the foregoing method embodiments can be implemented.

The description has been made with reference to various exemplary embodiments herein. However, a person skilled in the art would have appreciated that changes and modifications could have been made to the exemplary embodiments without departing from the scope herein. For example, various operation steps and assemblies for executing operation steps may be implemented in different ways according to a specific application or considering any number of cost functions associated with the operation of the system (for example, one or more steps may be deleted, modified or incorporated into other steps).

In addition, as understood by a person skilled in the art, the principles herein may be reflected in a computer program product on a computer-readable storage medium that is pre-installed with computer-readable program code. Any tangible, non-transitory computer-readable storage medium can be used, including magnetic storage devices (hard disks, floppy disks, etc.), optical storage devices (CD-ROM, DVD, Blu Ray disks, etc.), flash memories, and/or the like. These computer program instructions can be loaded onto a general-purpose computer, a dedicated computer, or other programmable data processing apparatus to form a machine, such that these instructions executed on a computer or other programmable data processing apparatus can generate an apparatus that implements a specified function. These computer program instructions can also be stored in a computer-readable memory that can instruct a computer or other programmable data processing apparatus to operate in a specific manner, such that the instructions stored in the computer-readable memory can form a manufactured product, including an implementation apparatus that implements a specified function. The computer program instructions may also be loaded onto a computer or other programmable data processing apparatus, such that a series of operating steps are executed on the computer or other programmable device to produce a computer-implemented process, such that the instructions executed on the computer or other programmable device can provide steps for implementing a specified function.

The disclosure has been described by using specific examples above, which are merely for the purpose of facilitating understanding of the disclosure and are not intended to limit the disclosure. For a person skilled in the technical field to which the disclosure belongs, several simple deductions, variations, or replacements may also be made according to the idea of the disclosure.

1-33. (canceled)

34. A monitoring method, comprising:
obtaining a physiological signal;
performing waveform detection on the physiological signal to determine a target waveform position sequence;
performing waveform classification on a physiological signal segment corresponding to the target waveform position sequence, to determine a waveform type of each physiological signal segment corresponding to the target waveform position sequence;
performing anomaly detection on classified physiological signal segments by using at least two preset anomaly detection methods, and generating a target alarm event sequence according to detection results of the at least two anomaly detection methods, wherein an alarm event in the target alarm event sequence is an alarm event determined according to an anomalous physiological signal segment in the classified physiological signal segments; and
outputting the target alarm event sequence.

35. The monitoring method of claim 34, wherein performing waveform detection on the physiological signal to determine a target waveform position sequence comprises:
performing waveform detection on the physiological signal by using the first waveform detection method, to determine a first waveform position sequence;
performing waveform detection on the physiological signal by using the second waveform detection method, to determine a second waveform position sequence; and
determining the target waveform position sequence according to the first waveform position sequence and the second waveform position sequence,
wherein the first waveform detection method and the second waveform detection method are different methods.

36. The monitoring method of claim 34, wherein performing waveform detection on the physiological signal to determine a target waveform position sequence comprises:
performing waveform detection on the physiological signal by using a first waveform detection method, to determine a third waveform position sequence; and
performing, by using a second waveform detection method, waveform detection on a physiological signal segment corresponding to the third waveform position sequence, to determine the target waveform position sequence,
wherein a sensitivity of the first waveform detection method is higher than a sensitivity of the second waveform detection method, and a specificity of the second waveform detection method is higher than a specificity of the first waveform detection method,
wherein the first waveform detection method and the second waveform detection method are different methods.

37. The monitoring method of claim 35, wherein determining the target waveform position sequence according to the first waveform position sequence and the second waveform position sequence comprises:
if a confidence level of the first waveform detection method is greater than a confidence level of the second waveform detection method, determining that the target waveform position sequence is the first waveform position sequence; or
if a confidence level of the first waveform detection method is less than or equal than a confidence level of the second waveform detection method, determining that the target waveform position sequence is the second waveform position sequence.

38. The monitoring method of claim 37, wherein the method further comprises:
updating the confidence level of the first waveform detection method according to a proportion of a number of confirmed waveform positions in the first waveform position sequence; and
updating the confidence level of the second waveform detection method according to a proportion of a number of confirmed waveform positions in the second waveform position sequence.

39. The monitoring method of claim 35, wherein determining the target waveform position sequence according to the first waveform position sequence and the second waveform position sequence comprises:
adding matched waveform positions in the first waveform position sequence and the second waveform position sequence to the target waveform position sequence; and/or
for any physiological signal segment in the physiological signal, when a first waveform position that is in the first waveform position sequence and corresponds to the physiological signal segment does not match a second waveform position that is in the second waveform position sequence and corresponds to the physiological signal segment, matching the physiological signal segment, the first waveform position, and the second waveform position with a historical waveform database, wherein the historical waveform database stores a correspondence between a physiological signal segment and a corresponding detected waveform position; and
adding a successful match in the first waveform position and the second waveform position to the target waveform position sequence; and determining a failed match in the first waveform position and the second waveform position as a false detection.

40. The monitoring method of claim 34, wherein performing waveform classification on a physiological signal segment corresponding to the target waveform position sequence, to determine a waveform type of each physiological signal segment corresponding to the target waveform position sequence comprises:
performing, by using a first waveform classification method, waveform classification on the physiological signal segment corresponding to the target waveform position sequence, to determine a first waveform type sequence;
performing, by using a second waveform classification method, waveform classification on the physiological signal segment corresponding to the target waveform position sequence, to determine a second waveform type sequence; and
determining the waveform type of each physiological signal segment corresponding to the target waveform position sequence according to the first waveform type sequence and the second waveform type sequence,
wherein the first waveform classification method and the second waveform classification method are different methods.

41. The monitoring method of claim 34, wherein performing waveform classification on a physiological signal segment corresponding to the target waveform position sequence, to determine a waveform type of each physiological signal segment corresponding to the target waveform position sequence comprises:
performing, by using a first waveform classification method, waveform classification on the physiological signal segment corresponding to the target waveform position sequence, to determine a third waveform type sequence; and
performing, by using a second waveform classification method, waveform classification on a physiological signal segment corresponding to the third waveform type sequence, to determine the waveform type of each physiological signal segment corresponding to the target waveform position sequence,
wherein a sensitivity of the first waveform classification method is higher than a sensitivity of the second waveform classification method, and a specificity of the second waveform classification method is higher than a specificity of the first waveform classification method;
wherein the first waveform classification method and the second waveform classification method are different methods.

42. The monitoring method of claim 41, wherein performing, by using the second waveform classification method, waveform classification on a physiological signal segment corresponding to the third waveform type sequence, to determine the waveform type of each physiological signal segment corresponding to the target waveform position sequence comprises:
for any physiological signal segment in the third waveform type sequence, classifying the physiological signal segment by using the second waveform classification method, to obtain a second waveform type, and determining a target waveform type of the physiological signal segment according to the second waveform type and a first waveform type that is obtained by classifying the physiological signal segment by using the first waveform classification method; and
determining the target waveform type sequence according to target waveform types of physiological signal segments in the third waveform type sequence.

43. The monitoring method of claim 40, wherein determining the waveform type of each physiological signal segment corresponding to the target waveform position sequence according to the first waveform type sequence and the second waveform type sequence comprises:
determining same waveform types in the first waveform type sequence and the second waveform type sequence as waveform types of corresponding physiological signal segments; and/or
for any physiological signal segment corresponding to the target waveform position sequence, when a first waveform type that is in the first waveform type sequence and corresponds to the physiological signal segment is different from a second waveform type that is in the second waveform type sequence and corresponds to the physiological signal segment, matching the physiological signal segment, the first waveform type, and the second waveform type with a historical waveform type database, wherein the historical waveform type database stores a correspondence between a physiological signal segment and a corresponding waveform type;
determining a successful match in the first waveform type and the second waveform type as a waveform type of a corresponding physiological signal segment; and
determining a failed match in the first waveform type and the second waveform type as a false classification.

44. The monitoring method of claim 34, wherein a first anomaly detection method and a second anomaly detection method in the at least two anomaly detection methods are different methods, and performing anomaly detection on classified physiological signal segments by using at least two preset anomaly detection methods, and generating a target alarm event sequence according to detection results of the at least two anomaly detection methods comprise:
performing anomaly detection on the classified physiological signal segments by using the first anomaly detection method, to generate a first alarm event sequence;
performing anomaly detection on the classified physiological signal segments by using the second anomaly detection method, to generate a second alarm event sequence; and
generating a target alarm event sequence according to the first alarm event sequence and the second alarm event sequence.

45. The monitoring method of claim 34, wherein a first anomaly detection method and a second anomaly detection method in the at least two anomaly detection methods are different methods, and performing anomaly detection on classified physiological signal segments by using at least two preset anomaly detection methods, and generating a target alarm event sequence according to detection results of the at least two anomaly detection methods comprise:
performing anomaly detection on the classified physiological signal segments by using the first anomaly detection method, to generate a third alarm event sequence; and
performing, by using the second anomaly detection method, anomaly detection on a physiological signal segment corresponding to the third alarm event sequence, to generate the target alarm event sequence,
wherein a sensitivity of the first anomaly detection method is higher than a sensitivity of the second anomaly detection method, and a specificity of the second anomaly detection method is higher than a specificity of the first anomaly detection method.

46. The monitoring method of claim 45, wherein performing, by using the second anomaly detection method, anomaly detection on a physiological signal segment corresponding to the third alarm event sequence, to generate the target alarm event sequence comprises:
for a physiological signal segment corresponding to any alarm event in the third alarm event sequence, detecting the physiological signal segment by using the second anomaly detection method, to obtain a second alarm event, and determining a target alarm event corresponding to the physiological signal segment according to the second alarm event and a first alarm event that is obtained by detecting the physiological signal segment by using the first anomaly detection method; and
determining the target alarm event sequence according to target alarm events that are in the third alarm event sequence and correspond to physiological signal segments.

47. The monitoring method of claim 44, wherein generating a target alarm event sequence according to the first alarm event sequence and the second alarm event sequence comprises:
determining that the target alarm event sequence is the first alarm event sequence, when a confidence level of the first anomaly detection method is greater than a confidence level of the second anomaly detection method; or
determining that the target alarm event sequence is the second alarm event sequence, when a confidence level of the first anomaly detection method is less than or equal to a confidence level of the second anomaly detection method.

48. The monitoring method of claim 47, wherein the method further comprises:
updating the confidence level of the first anomaly detection method according to a proportion of a number of confirmed alarm events in the first alarm event sequence; and/or
updating the confidence level of the second anomaly detection method according to a proportion of a number of confirmed alarm events in the second alarm event sequence.

49. The monitoring method of claim 43, wherein generating a target alarm event sequence according to the first alarm event sequence and the second alarm event sequence comprises:
adding matched alarm events in the first alarm event sequence and the second alarm event sequence to the target alarm event sequence; and/or
for any physiological signal segment in the classified physiological signal segments, when a first alarm event that is in the first alarm event sequence and corresponds to the physiological signal segment does not match a second alarm event that is in the second alarm event sequence and corresponds to the physiological signal segment, matching the physiological signal segment, the first alarm event, and the second alarm event with a historical alarm database, wherein the historical alarm database stores a correspondence between a physiological signal segment and a corresponding detected alarm event;
adding a successful match in the first alarm event and the second alarm event to the target alarm event sequence; and
determining a failed match in the first alarm event and the second alarm event as a false alarm.

50. The monitoring method of claim 44, wherein
when one of the first anomaly detection method and the second anomaly detection method is to perform anomaly detection on the physiological signal segment based on a preset alarm threshold according to at least one of a waveform type, waveform start and end points, a heart rate, an amplitude, and an interval of the physiological signal segment, the other method is to perform anomaly detection on the physiological signal segment by using a pre-trained artificial intelligence alarm model, wherein the artificial intelligence alarm model is trained based on a physiological signal segment annotated with an alarm event.

51. The monitoring method of claim 34, wherein performing anomaly detection on classified physiological signal segments by using at least two preset anomaly detection methods, and generating a target alarm event sequence according to detection results of the at least two anomaly detection methods comprise:
performing anomaly detection on the classified physiological signal segments by using the at least two preset anomaly detection methods, to generate an alarm event set;
for any alarm event in the alarm event set, obtaining a plurality of pieces of priority-related characteristic information of the alarm event;
respectively inputting the plurality of pieces of characteristic information to a plurality of corresponding pre-trained alarm priority models, to obtain a plurality of sub-priorities of the alarm event;
determining a target priority of the alarm event according to the plurality of sub-priorities of the alarm event; and
sorting alarm events in the alarm event set according to target priorities of the alarm events in the alarm event set, to obtain the target alarm event sequence.

52. The monitoring method of claim 34, wherein before performing waveform detection on the physiological signal, the method further comprises:
determining a signal quality index of the physiological signal according to at least one of an amplitude, a slope, and a power spectrum of the physiological signal.

53. The monitoring method of claim 52, wherein analyzing the physiological signal to obtain a signal quality index of the physiological signal comprises:
inputting the physiological signal to a pre-trained artificial intelligence signal quality evaluation model, to obtain the signal quality index of the physiological signal, wherein the artificial intelligence signal quality evaluation model is trained based on a physiological signal annotated with a signal quality index.
